(19) United States
(12) Patent Application Publication
(10) Pub. No.: US 2025/0352782 A1
(43) Pub. Date: Nov. 20, 2025

(54) METHOD AND APPARATUS FOR CONVERTING BETWEEN RECIPROCATING TRANSLATIONAL MOTION AND ROTATIONAL MOTION

(71) Applicant: David M. Lancisi, Folsom, CA (US)

(72) Inventor: David M. Lancisi, Folsom, CA (US)

(21) Appl. No.: 18/665,206

(22) Filed: May 15, 2024

(51) Int. Cl. A61M 60/882; H01F 7/02

(52) U.S. Cl. CPC A61M 60/882; H01F 7/0242; A61M 2206/16; A61M 2210/125

(57) ABSTRACT

Motion conversion apparatus for converting rotational motion to translational motion is described. The apparatus includes a rotating member magnetically coupled to a translating member. The translating member translates in response to rotation of the rotating member. The translating member is substantially constrained from rotational movement in one embodiment. The rotational member is substantially constrained from translational movement in one embodiment. In various embodiments, the motion conversion apparatus is incorporated to form a pumping apparatus. The pumping apparatus may be adapted for use as a heart ventricle replacement or alternatively for a total artificial heart. In one embodiment the pumping apparatus includes a deformable pumping chamber to which the translating member is coupled. In response to rotation of the rotating member, the translating member translates to deform or restore the pumping chamber causing fluid to be expelled or taken into the pumping chamber.

### FIELD OF THE INVENTION

The present invention relates to method and apparatus for converting between rotational motion and translational motion, generation of reciprocating translational motion, and application to pumps.

### BACKGROUND

A slider-crank converts mechanical rotary motion to translational motion or vice-versa. Slider-cranks are used to convert rotary motion to translational motion, for example, in a reciprocating piston pump. A reciprocating piston engine relies on a slider-crank to convert translatory motion to rotary motion.

Another apparatus for converting mechanical rotary motion to translational motion utilizes a threaded lead screw and a grooved nut which translates along the lead screw as the lead screw is rotated. The nut is typically fastened to the object to be moved. A variation on the lead screw and nut is a ball screw or grooved screw. The nut contains ball bearings which travel the grooves of a grooved screw as it is rotated thus causing translational motion of the nut. Other apparatus for converting rotary motion to translational motion rely on gears such as worm drives for the conversion.

The slider-crank, lead/ball screw, and worm drives experience friction and wear and tear due to the contact between the rotating and the translating components. For applications such as pumps where hermicity may be required, shaft seals must be implemented. The shaft seals also experience degradation due to the contact between the rotating and translating components.

### SUMMARY

One embodiment of a motion conversion apparatus includes a rotating member and a translating member. The rotating member and translating member are magnetically coupled. The translating member translates in response to rotation of the rotating member. The rotating member carries a plurality m of permanent magnetic poles. The translating member carries a plurality n of permanent magnetic poles. The variables m and n are even integers. In one embodiment, m=n. In one embodiment m, n=2. In another embodiment, m, n=b*2 where b is an integer greater than 1. In other embodiments, m≠n.

One embodiment of a pump apparatus includes a rotating member and a translating member. The apparatus includes a pumping chamber. The rotating member and the translating member are magnetically coupled. The translating member translates to displace fluid in the pumping chamber in response to rotating of the rotating member. The translating member carries a plurality n of permanent magnetic poles. The variables m and n are even integers. In one embodiment, m=n. In one embodiment m, n=2. In another embodiment, m, n=b*2 where b is an integer greater than 1. In other embodiments, m≠n.

Another embodiment of a pump apparatus includes a rotating member and a translating member. The apparatus includes a deformable pumping chamber. The translating member is coupled to the pumping chamber. The rotating member and the translating member are magnetically coupled. The translating member translates to displace fluid in the pumping chamber in response to rotation of the rotating member. The rotating member carries a plurality m of permanent magnetic poles. The translating member carries a plurality n of permanent magnetic poles. The variables m and n are even integers. In one embodiment, m=n. In one embodiment m, n=2. In another embodiment, m, n=b*2 where b is an integer greater than 1. In other embodiments, m≠n. In one embodiment the deformable pumping chamber has a spherical cap form factor. In one embodiment the fluid is blood. In one embodiment, the pump apparatus is adapted to perform as a ventricle of a heart. In another embodiment, the pump apparatus is adapted to perform as a total artificial heart.

Other features and advantages of the present invention will be apparent from the accompanying drawings and from the detailed description that follows below.

### BRIEF DESCRIPTION OF THE DRAWINGS

The present invention is illustrated by way of example and not limitation in the figures of the accompanying drawings in which like references indicate similar elements and in which:

FIG. 1 illustrates a cross-sectional view of one embodiment of an apparatus for converting between rotational motion and translational motion.

FIG. 2 illustrates a perspective view of the apparatus of FIG. 1.

FIG. 3A illustrates a perspective view of the magnet arrays carried by the rotating member and the translating member of FIG. 2 with m=n=2.

FIG. 3B illustrates a top view of the translating member of FIG. 3A juxtaposed a bottom view of the rotating member of FIG. 3A.

FIG. 4 illustrates one alternative embodiment of the magnet arrays carried by the rotating and translating members with m=n=4.

FIG. 5 illustrates a cross-section of an alternative embodiment of an apparatus for converting rotational motion to translating motion.

FIG. 6 illustrates a functional diagram for the operation of a human heart.

FIG. 7 illustrates a functional diagram for the operation of a human heart with ventricles replaced by a pump incorporating a motion conversion apparatus.

FIG. 8A illustrates a top view of an alternative embodiment of an apparatus for converting rotational motion to translational motion integrated into a pumping dome.

FIG. 8B illustrates a perspective view of the apparatus of FIG. 8A.

FIG. 9 illustrates a functional diagram for replacing the pumping function of the human heart with pumps incorporating a motion conversion apparatus.

FIG. 10A illustrates a top view of one embodiment of an apparatus for converting rotational motion to translational motion integrated into a dome-shaped pumping chamber.

FIG. 10B illustrates a perspective view of the apparatus of FIG. 10A.

For simplicity and clarity of illustration, elements illustrated in the drawings have not necessarily been drawn to scale. For example, the dimensions of some of the elements are exaggerated relative to other elements for clarity. Further, where considered appropriate, reference numerals have been repeated among the drawings to indicate corresponding or analogous elements or multiple instances of the same element.

### DETAILED DESCRIPTION

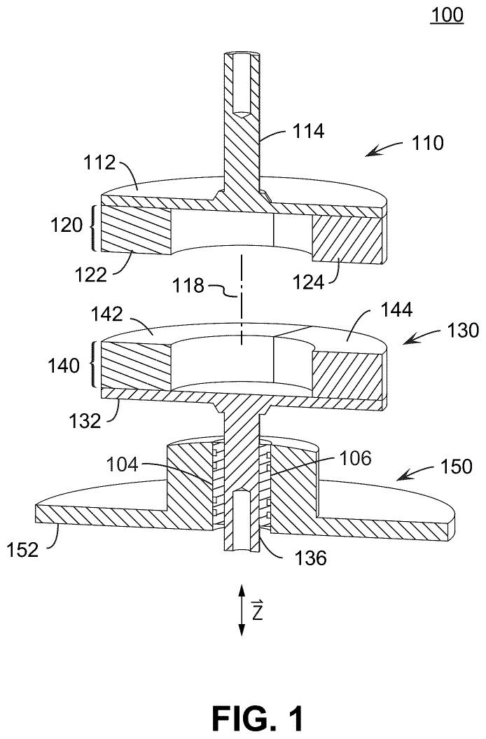

FIG. 1 illustrates a cross-sectional view of one embodiment of an apparatus 100 for converting between rotational motion and translational motion. The apparatus includes a rotating member 110 and a translating member 130.

In the illustrated embodiment, the rotating member 110 includes a disk 112 coupled to a shaft 114 for driving the rotating member. The translating member 130 includes a disk 132 coupled to a translating shaft 136.

The rotating member carries a plurality, m, of permanent magnetic poles 122, 124 collectively identified as array 120. The translating member likewise carries a plurality, n, of permanent magnetic poles 142, 144 collectively identified as array 140.

The numbers m and n are even integers. Each plurality of magnetic poles 120, 140 is arranged in substantially circular form on the corresponding disk 112, 132. In the illustrated embodiment, discrete permanent magnets 122, 124, 142, 144 implement the magnetic poles.

In one embodiment, m=n. In other embodiments, m=n. In the illustrated embodiment of FIG. 1, m=n=2.

The permanent magnetic poles carried by each member 110, 130 are positioned to lie on the periphery of a circle. The magnetic poles carried by each member are arranged such that adjacent or sequential poles on the same member have opposite polarities—the polarity of magnetization alternates around the circles.

The translating member is constrained to prevent rotation. The rotational member may be constrained to prevent translation. The two members are placed proximate to but not so close as to make physical contact with each other during operation.

In the illustrated embodiment, the rotating member has a disk-shaped face 112 lying in a plane orthogonal to the axis of rotation 118. The translating member has a disk-shaped face 132 lying in a plane orthogonal to the path of translation 118. The members are positioned such that the respective disk-shaped faces oppose each other. In the illustrated embodiment, the axis of rotation of the rotating member is substantially axially aligned and identical to the translation path 118 of the translating member.

In one embodiment, the shaft 136 of the translating member 130 is radially supported by a linear bearing or a bushing 106 disposed within a bore 104 of a retaining assembly 150. The bushing permits the shaft of the translating member to translate along translation path 118. Although not illustrated in this figure, the translating member 130 is constrained to prevent rotation. Thus the translating member can only translate (i.e., not rotate) along or about the path 118.

The rotating member and translating member are magnetically coupled. The magnetic coupling enables transmission of force without physical contact. The translating member 130 translates along the path of translation 118 in response to rotation of the rotating member 110 or vice-versa.

The rotating member 110 and translating member 130 are positioned such that their respective magnetic arrays 120, 140 are co-axially aligned with each other about the axis of rotation 118. The rotating member and translating member are positioned sufficiently far apart to prevent the translating member from contacting the rotating member at the extreme ends of the path of translation.

In one application, a torque is applied to the rotating member to drive the translating member. As the rotating member 110 rotates about axis 118, its magnetic array 120 will reach an angular displacement or position θ relative to the position of the magnetic array 140 of the translating member for which the magnetic coupling between the members is such that forces of repulsion overwhelm any forces of attraction. The repulsive magnetic force causes translation of the translating member 130 away from the rotating member 110 along the path of translation 118.

As the rotating member continues to rotate, the angular displacement θ or angular position of the magnetic arrays 120, 140 with respect to each other changes until the magnetic coupling between the members is such that the forces of attraction overwhelm repulsive forces. The attractive magnetic force causes translation of the translating member towards the rotating member along the path of translation 118. Rotation of the rotating member causes reciprocating translational movement of the translating member.

In one application, force is applied to the translating member to drive the rotational member. Reciprocating translation of the translating member will cause rotation of the rotating member.

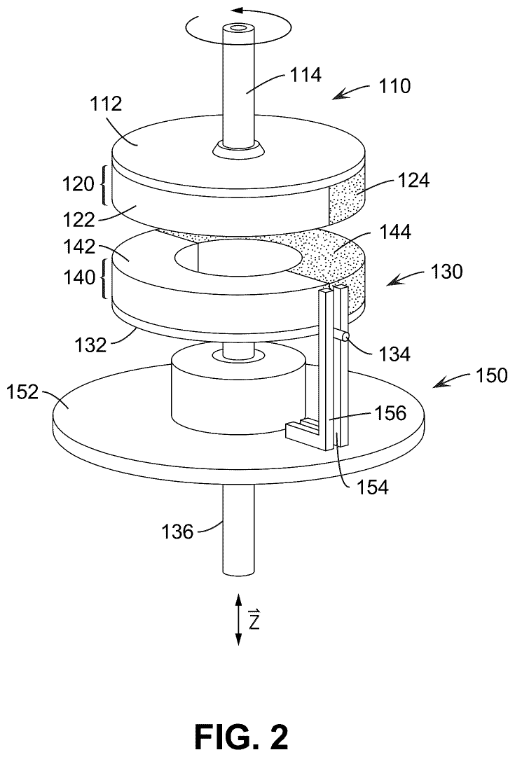

FIG. 2 illustrates a perspective view of the apparatus of FIG. 1. The apparatus includes a rotating member 110 and a translating member 130.

The rotating member has a disk-shaped face 112 lying in a plane orthogonal to the axis of rotation 118. The translating member has a disk-shaped face 132 lying in a plane orthogonal to the path of translation 118. The members are positioned such that the respective disk-shaped faces oppose each other. In the illustrated embodiment, the axis of rotation of the rotating member is substantially axially aligned and identical to the translation path 118 of the translating member.

Translating member 130 includes an anti-rotation feature. In the illustrated embodiment, translating member 130 includes an anti-rotation pin 134. Anti-rotation pin 134 is disposed between tines 156 of an anti-rotation bracket 154 mounted to a disk-shaped surface 152 of the retaining assembly. The anti-rotation pin and bracket permit the translating member to translate along the path of translation while constraining the translating member from rotating about the axis of rotation. The shaft 136 of the translating member 130 is radially supported by a linear bearing or a bushing 158 disposed within a bore of the retaining assembly 150.

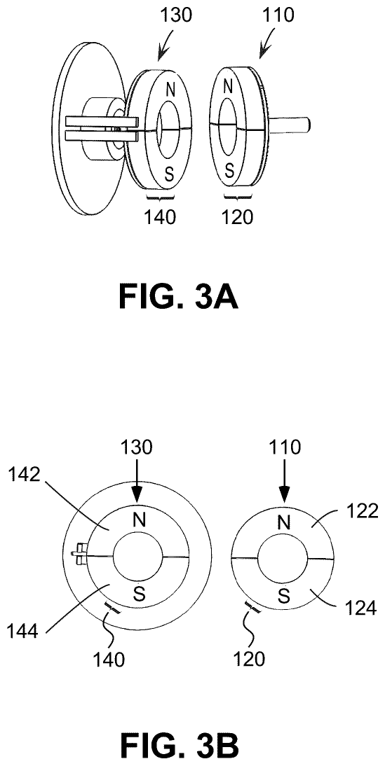

FIG. 3A illustrates a perspective view of the magnet arrays 120, 140 carried by the rotating member 110 and the translating member 130 of FIG. 2 with m=n=2. FIG. 3B illustrates a top view of the translating member of FIG. 3A juxtaposed a bottom view of the rotating member of FIG. 3A.

The array 120 carried by the rotating member 110 is formed by discrete arcuate permanent magnet segments 122, 124 forming a circle or ring about the axis of rotation. The array 140 carried by the translating member 130 is formed by discrete arcuate permanent magnet segments 142, 144 forming a circle or ring about the translating shaft. The permanent magnet segments are axially magnetized. The “N” and “S” indicate the orientation of the magnetic pole vectors. The magnetic poles of the rotating member array are parallel to the axis of rotation of the rotating member. The magnetic poles of the translating member array are parallel to the longitudinal axis of the translating shaft (i.e., the path of translation of the translating member).

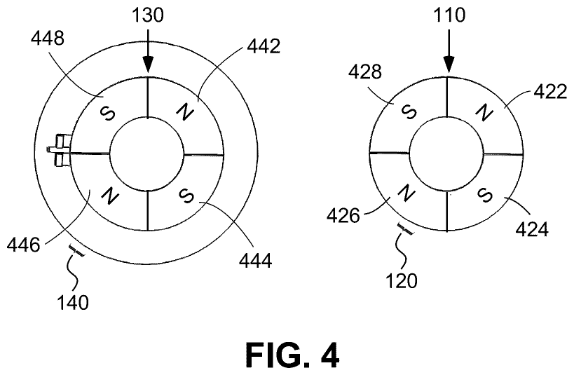

FIG. 4 illustrates an alternative embodiment of the magnet arrays carried by the rotational and translational members with m=n=4 (i.e., b=2). The array 120 carried by the rotating member 110 is formed by discrete arcuate permanent magnet segments 422, 424, 426, 428 forming a circle or ring about the axis of rotation. The permanent magnet segments are axially magnetized such that the magnetic poles are parallel to the axis of rotation of the rotating member. The “N” and “S” indicate the orientation of the magnetic pole vectors.

The array 140 carried by the translating member 130 is formed by discrete arcuate permanent magnet segments 442, 444, 446, 448 forming a circle or ring about the path of translation. The permanent magnet segments are axially magnetized such that the magnetic poles are parallel to the axis of rotation of the rotating member when in operation. The magnetic poles are also parallel to the path of translation of the translating member.

The number of pole-pairs affect that frequency of reciprocation of the translating member with respect to the frequency of rotation of the rotating member. In other words, f=b ω, where f is the reciprocating frequency of the translating member, b is the number of pole-pairs (or number of poles divided by two) carried by each member when m=n, and ω is the rotational speed of the rotating member. A 360 degree rotation of the rotating member will result in one full cycle of translation, i.e., translation to the distal end of the path of translation returning to the proximate end of the path of translation relative to the rotating member when m=n=2 (i.e., b=1). The frequency of reciprocation of the translating member will be twice the frequency of rotation of the rotating member when m=n=4 (i.e., b=2).

In alternative embodiments, form factors other than arcuate segments may be utilized for the permanent magnet assemblies. The assemblies may comprise a plurality of permanent magnets with cylindrical, rectangular prism, or other form factors arranged about the axis of rotation and translating shaft.

Instead of implementing each magnetic pole with a discrete permanent magnet, a permanent magnet may be magnetized to have multiple polarities. For example, instead of two discrete arcuate segments as illustrated in FIG. 3A or four discrete arcuate segments as illustrated in FIG. 4, a monolithic cylindrical ring may be magnetized to provide the desired number of poles. Thus in one embodiment the permanent magnet assemblies carried by each member comprises a single ring.

The permanent magnet arrays of FIG. 3A and FIG. 4 illustrate alternating magnetic polarities 180 degrees out of phase adjacent each other to generate an alternating polarity axial magnetic field. In alternative embodiments, the permanent magnet arrays may use discrete magnets or monolithic magnets magnetized to generate a magnetic field other than alternating opposite polarities. For example, in one embodiment, the polarity varies sinusoidally.

The strength of the magnetic coupling between the rotating and translating members varies inversely with the square of the distance between them. The magnetic coupling is weakest when the translating member is positioned at the distal end of the path of translation and strongest at the proximal end.

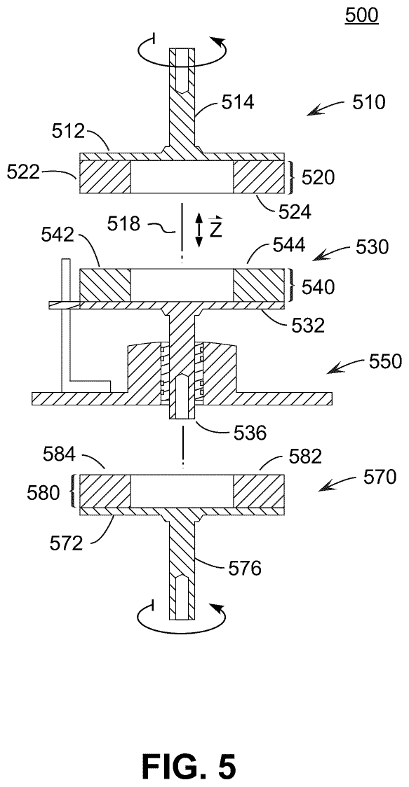

FIG. 5 illustrates a cross-sectional view of an alternative embodiment of an apparatus for converting between rotational and translational motion. The apparatus includes first rotating member 510, and a translating member 530, and a second rotating member 570. The first rotating member 510 includes a disk 512 coupled to a shaft 514 for driving the first rotating member. The translating member 530 includes a disk 532 coupled to a translating shaft 536. The second rotating member 570 includes a disk 572 coupled to a shaft 574 for driving the second rotating member.

The first rotating member carries a plurality, m, of permanent magnetic poles 522, 524 collectively identified as array 520. The second rotating member carries a plurality, m, of permanent magnetic poles 582, 584 collectively identified as array 580. The translating member carries a plurality, n, of permanent magnetic poles 542, 544 collectively identified as array 540.

The numbers m and n are even integers. In one embodiment, m=n. In other embodiments, m≠n. In the illustrated embodiment of FIG. 5, m=n=2. Each plurality of magnetic poles 520, 540, 580 is arranged in substantially circular form on the corresponding disk 512, 532, 572. In the illustrated embodiment, discrete permanent magnets 522, 524, 542, 544, 582, 584 implement the magnetic poles.

The permanent magnetic poles carried by each member 510, 530, 570 are positioned to lie on the periphery of a circle centered on the axis of rotation. The magnetic poles carried by each member are arranged such that adjacent or sequential poles on the same member have opposite polarities—the polarity of magnetization alternates around the circles. Variations for the polarity of magnetization and magnetic poles as previously described may likewise be applied.

The translating member 530 is constrained to prevent rotation. The rotational members 510, 570 may be constrained to prevent translation. The translating member is disposed between the two rotating members but not so close as to make physical contact during operation.

In the illustrated embodiment, the first rotating member has a disk-shaped face 512 lying in a plane orthogonal to the axis of rotation 518. The translating member has a disk-shaped face 532 lying in a plane orthogonal to the path of translation 518. The second rotating member has a disk-shaped fact 572 lying in a plane orthogonal to the axis of rotation 518. The members are positioned such that the respective disk-shaped faces oppose each other and are co-axially aligned. In the illustrated embodiment, the axis of rotation of the first and second rotating members are substantially the same and axially aligned with the path of translation 518 of the translating member.

The first rotating member 510, second rotating member 570, and translating member 530 are positioned such that their respective magnetic arrays are coaxially aligned with each other about the axis of rotation 518. As the first rotating member 510 rotates about axis 518, its magnetic array 520 will reach an angular displacement or position θ relative to the position of the magnetic array 540 of the translating member for which the magnetic coupling is net repulsive. The repulsive magnetic force causes translation of the translating member 530 away from the first rotating member 510 along the path of translation 518. As the translating member translates away from the first rotating member 510, the magnetic coupling between them is weaker. The magnetic force of repulsion when the translating member is proximal to the first rotating member is greater than the magnetic force of attraction when the translating member is distal to the first rotating member.

If the translation distance is short, the asymmetry in magnetic force may be inconsequential. However, the asymmetry may be consequential for longer translation paths. The second rotating member 570 ameliorates the asymmetry by providing a magnetic field opposite that of the first rotating member. The second rotating member exerts a repulsive magnetic force on the translating member when the first rotating member is exerting an attractive magnetic force. The second rotating member exerts an attractive magnetic force on the translating member when the first rotating member is exerting a repulsive magnetic force. In one embodiment, the orientation of the poles of magnetic array 520 carried by the first rotating member relative to the poles of the magnetic array 580 of the second rotating member is 180 degrees.

The motion conversion apparatus described in the previous embodiments relied on rotating magnetic fields. Although rotating magnetic fields can be implemented through the use of permanent magnets carried by a rotating member, rotating magnetic fields can also be generated electrically. In particular, sequentially energizing conductive coils produces a rotating magnetic field.

The motion conversion apparatus may be applied to implement a pump. Referring to FIG. 2, the translating member may be housed within a pump housing or otherwise coupled to a pumping chamber coupled to receive the fluid to be pumped and with an output to eject the pumped fluid. In one application, such pump is utilized to perform as a blood pump to replace the function of a ventricle of a human heart.

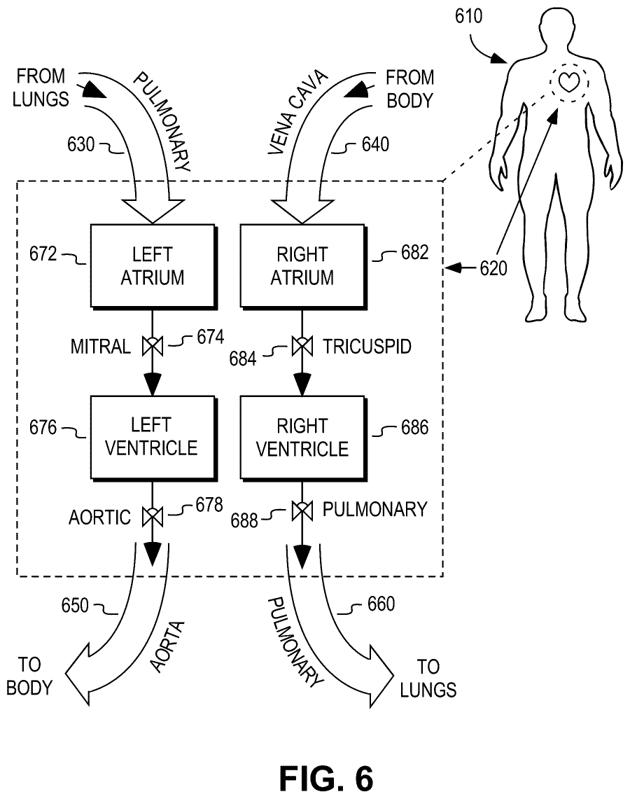

FIG. 6 illustrates a functional diagram of a human heart. The human heart 620 is located in the upper torso of the human body 610. The heart has four chambers: left atrium 672, right atrium 682, left ventricle 676, and right ventricle 686.

The left atrium 672 receives oxygenated blood from the lungs via the pulmonary vein 630. Oxygenated blood in the left atrium is transferred to the left ventricle 676 through the mitral valve 674. The left ventricle 676 pumps the oxygenated blood through the aortic valve 678 to the rest of the body via an artery known as the aorta. Although not illustrated in detail, the aorta connects to a network of smaller arteries and capillaries for distribution of the blood throughout the body.

Oxygen depleted blood from the body is received into the right atrium 682 via the vena cava vein 640. The oxygen depleted blood is transferred to the right ventricle 686 through the tricuspid valve 684. The right ventricle 686 pumps the oxygen depleted blood to the lungs via the pulmonary artery 660. The blood is oxygenated by the lungs and returns to the heart via the pulmonary vein 630 where the cycle continues. The mitral, aortic, tricuspid, and pulmonary valves are one-way valves. Contractions of the ventricles cause the pumping action.

The heart's pumping efficiency can suffer from a variety of causes including disease, damage, or birth defect. Some people are born with a single ventricle. Others may suffer damage to ventricles as a result of a heart attack. The heart may be weakened as a result of high blood pressure, obesity, and other maladies. In some cases, surgery may employed to repair the damage. In other cases, repair is not an option and the functionality must be replaced if the patient is to survive. Market solutions for replacing the functionality has appeared as pneumatic pumping chambers which are surgically implanted. The patient is tethered to an air compressor via hoses that pass through the wall of the torso. Quality of life for the patient may be improved by a solution that does not require protruding hoses and perpetual tethering to an air compressor.

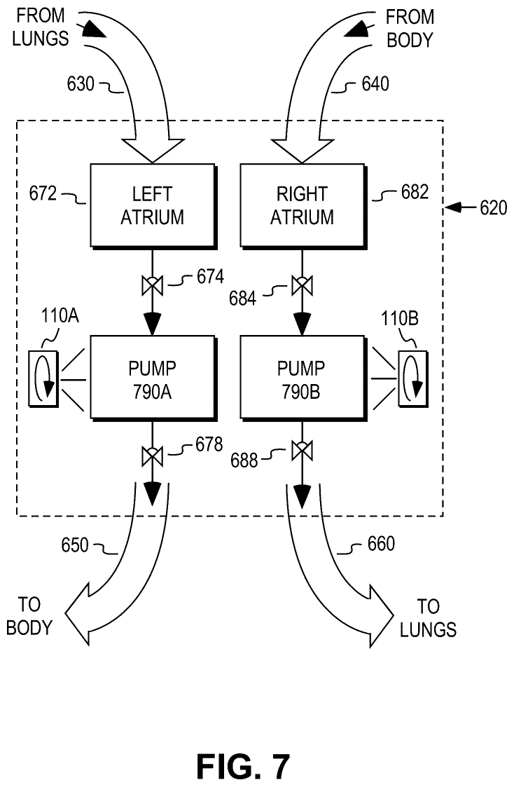

FIG. 7 illustrates a functional diagram for the operation of a human heart with ventricles replaced by a pump incorporating the motion conversion apparatus. Comparing FIG. 6 and FIG. 7, the left ventricle 676 is replaced with pump 790A and the right ventricle is replaced with pump 790B. The pump functional blocks are implemented with the translational member component of the rotational to translational motion conversion apparatus. The translational components of the pumps 790A, 790B are magnetically or electromagnetically coupled to the rotating members 110A, 110B. The rotational members provide the rotating magnetic driving force driven electrically or electromechanically (e.g., motors). The form factor of the pumps is such that the pumps and rotational members can be implanted within the torso. Although a source of electrical power is required to power the rotational members, the power can be provided by rechargeable batteries which enables greater freedom of movement for the patient. The patient avoids the noise and tethering of pneumatic devices. To the extent a physical conductor needs to pass through the outer torso wall, the conductors can be much smaller and less intrusive than the pneumatic hoses.

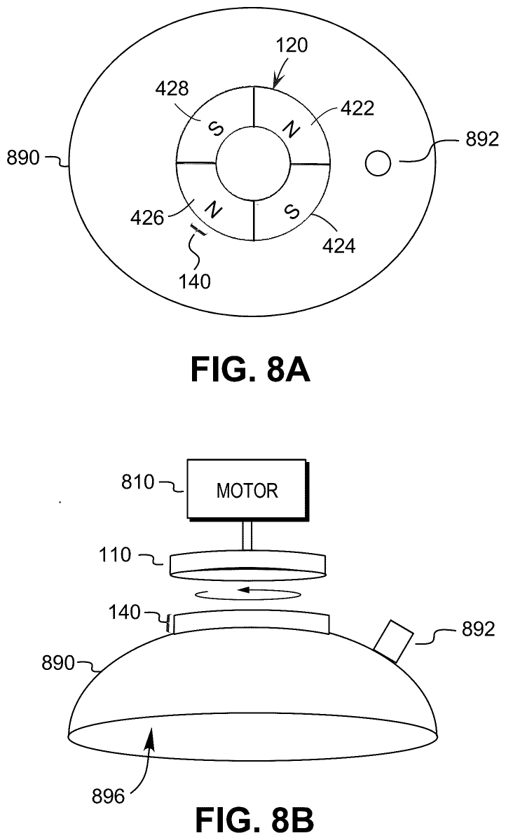

FIG. 8A illustrates a top view of an alternative embodiment of an apparatus for converting rotational motion to translational motion integrated into a pumping dome. The dome is deformable such that it forms a deformable pumping chamber.

In the illustrated embodiment, the deformable pumping chamber 890 has a form factor substantially similar to a spherical cap. The pumping chamber includes an input or intake port 892. The translating member includes magnet array 140 which is attached to the deformable pumping chamber 890. The dome or deformable pumping chamber constrains the translating member from rotation. No shaft is required for the translating member to operate the deformable pumping chamber. In use, the base of the dome is surgically sewn to the appropriate portion of the heart so as to form an artificial ventricle.

FIG. 8B illustrates a perspective view of the dome 890 and translating member 140 of FIG. 8A and the rotating member 110. The rotating member 110 is placed proximate to and co-axially aligned to the translating member 140. The rotating member is magnetically coupled to the translating member. Rotating the rotating member causes the translating member to be alternately repelled and attracted.

When repelled, the translating member causes a deformation of the pumping chamber so as to expel fluid (e.g., blood) from the chamber. The base of the deformable pumping chamber is open to its interior 896. When surgically implanted in a heart, the pumping chamber expels blood through the aortic or pulmonary arteries. As the rotation of the rotating member reaches an angle such that the translating member 140 is attracted to the rotating member, the translating member moves towards the rotating member causing blood to enter the pumping chamber via the pump input port 892. The blood is sourced from the associated atrium through a one-way valve. The rotating magnetic field of the rotating member is achieved electromechanically via a motor 810 in one embodiment. The rotation of the rotating member thus results in reciprocating translation of the translating member. In response to reciprocating translation of the translating member the fluid is alternately drawn into and then expelled from the pumping chamber.

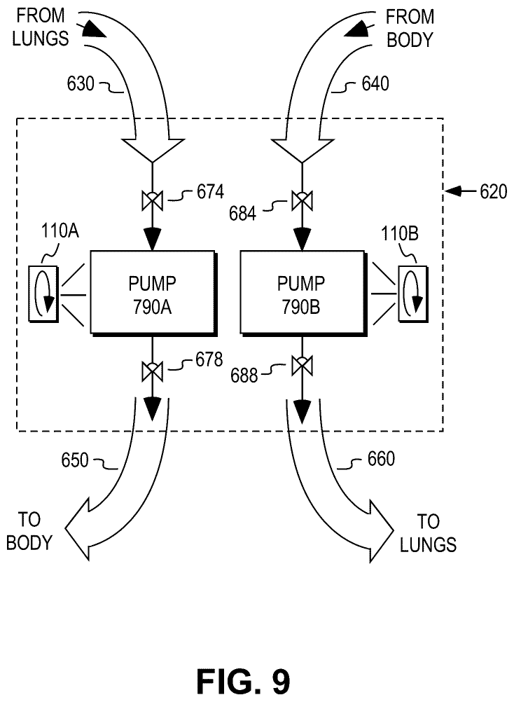

FIG. 9 illustrates a functional diagram for replacing the pumping function of the human heart with pumps incorporating a motion conversion apparatus. The valves 674, 678, 684, 688 provide the functionality of the mitral, aortic, tricuspid, and pulmonary valves. In various embodiments, one or more of valves 678, 678, 684, 688 is a mechanical valve or a biological valve other than the recipient's original biological valve.

The functional diagram of FIG. 9 does not rely on atria and the ventricles have been replaced by pumps 790A, 790B incorporating the motion conversion apparatus. This embodiment corresponds to a total artificial heart because it does not rely upon any biological heart tissue.

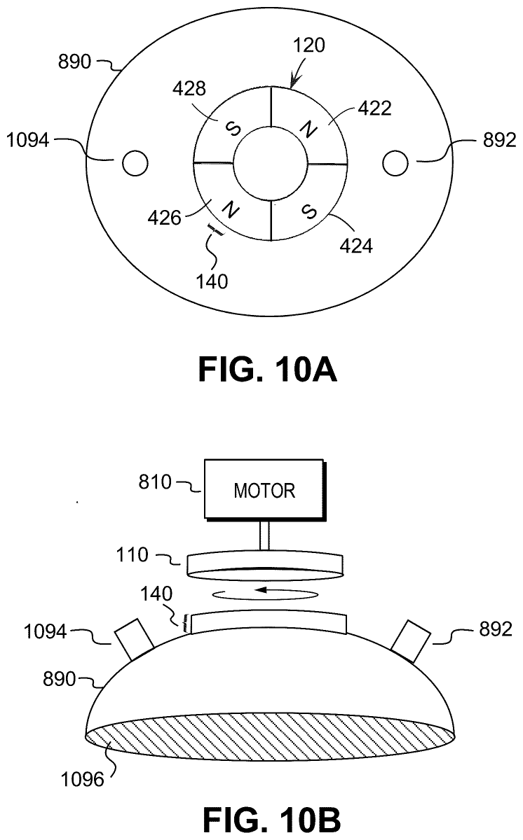

FIG. 10A illustrates a top view of one embodiment of an apparatus for converting rotational motion to translational motion integrated into a dome-shaped pumping chamber 890.

FIG. 10B illustrates a perspective view of the apparatus of FIG. 10A including the dome 890, translating member 140, and the rotating member 110. The pumping chamber includes an inlet port 892 for drawing blood into the pumping chamber. The pumping chamber includes an outlet port 1094 for expelling blood from the pumping chamber. The base of the dome-shaped pumping chamber 890 is not open to its interior. A wall 1096 seals the base. With the exception of the inlet and outlet ports, the dome-shaped pumping chamber 890 forms a closed chamber. When in use as a total artificial heart, fluid ingress to the pumping chamber via the inlet port 892 is gated by a valve 674 (or 684). Similarly, fluid egress from the pumping chamber via the outlet port 1094 is gated by a valve 678 (or 688).

The rotating member 110 is placed proximate to and co-axially aligned to the translating member 140. The rotating member is magnetically coupled to the translating member. Rotating the rotating member causes the translating member to be alternately repelled and attracted. When repelled, the translating member causes a deformation of the pumping chamber 890 so as to expel fluid (e.g., blood) from the chamber through the outlet port 1094. The base of the deformable pumping chamber is not open to its interior. The base is sealed by wall 1096. As the rotation of the rotating member reaches an angle such that the translating member 140 is attracted to the rotating member, the translating member moves towards the rotating member causing blood to enter the pumping chamber via the pump input port 892. The rotating magnetic field of the rotating member is achieved electromechanically via a motor 810 in one embodiment.

Although the invention has been described and illustrated with reference to the specific embodiments, it is not intended that the invention be limited to the illustrative embodiments. Those skilled in the art will recognize that modifications and variations may be made without departing from the spirit and scope of the invention. Therefore, it is intended that this invention encompass all of the variations and modification as fall within the scope of the appended claims.

What is claimed is:

1. A motion conversion apparatus, comprising:
a rotating member; and
a translating member, wherein the rotating member and translating member are magnetically coupled, wherein the translating member translates in response to rotation of the rotating member.

2. The apparatus of claim 1 wherein the rotating member carries a plurality, m, of permanent magnetic poles, wherein the translating member carries a plurality, n, of permanent magnetic poles, wherein m and n are even integers.

3. The apparatus of claim 2 wherein m=n.

4. The apparatus of claim 2 wherein m≠n.

5. The apparatus of claim 2 wherein the m permanent magnetic poles are arranged substantially in circular form about a face of the rotating member, wherein the n permanent magnetic poles are arranged substantially in circular form about a face of the translating member.

6. The apparatus of claim 1 wherein the translating member is substantially constrained from rotational movement, wherein the rotational member is substantially constrained from translational movement.

7. A pump apparatus, comprising:
a rotating member;
a translating member; and
a pumping chamber, wherein the rotating member and translating member are magnetically coupled, wherein the translating member translates to expel fluid from the pumping chamber in response to rotation of the rotating member.

8. The apparatus of claim 7 wherein the rotating member carries a plurality, m, of permanent magnetic poles, wherein the translating member carries a plurality, n, of permanent magnetic poles, wherein m and n are even integers.

9. The apparatus of claim 8 wherein m=n.

10. The apparatus of claim 8 wherein m≠n.

11. The apparatus of claim 8 wherein the m permanent magnetic poles are arranged substantially in circular form about a face of the rotating member, wherein the n permanent magnetic poles are arranged substantially in circular form about a face of the translating member.

12. The apparatus of claim 7 wherein the translating member is substantially constrained from rotational movement, wherein the rotating member is substantially constrained from translational movement.

13. The apparatus of claim 7 wherein in response to reciprocating translation of the translating member the fluid is alternately drawn into and then expelled from the pumping chamber.

14. A pump apparatus, comprising:
a rotating member;
a translating member, wherein the rotating member and translating member are magnetically coupled; and
a deformable pumping chamber, wherein the translating member is coupled to the pumping chamber, wherein the translating member translates to displace fluid in the pumping chamber in response to rotation of the rotating member.

15. The apparatus of claim 14 wherein the rotating member carries a plurality, m, of permanent magnetic poles, wherein the translating member carries a plurality, n, of permanent magnetic poles, wherein m and n are even integers.

16. The apparatus of claim 15 wherein m=n.

17. The apparatus of claim 15 wherein m≠n.

18. The apparatus of claim 15 wherein the m permanent magnetic poles are arranged substantially in circular form about a face of the rotating member, wherein the n permanent magnetic poles are arranged substantially in circular form about a face of the translating member.

19. The apparatus of claim 14 wherein the pumping chamber has a substantially spherical cap form factor when not deformed.

20. The apparatus of claim 14 wherein the fluid is blood.

21. The apparatus of claim 14 adapted for use as a total artificial heart.
